(19) United States
(12) Patent Application Publication
(10) Pub. No.: US 2014/0235576 A1
(43) Pub. Date: Aug. 21, 2014

(54) PYRROLIDINE-2-CARBOXYLIC ACID DERIVATIVES AS IGLUR ANTAGONISTS

(71) Applicant: Kobenhavns Universitet, Copenhagen K (DK)

(72) Inventor: Lennart BUCH, Copenhagen SV (DK)

(73) Assignee: Kobenhavns Universitet, Copenhagen K (DK)

(21) Appl. No.: 14/181,169

(22) Filed: Feb. 14, 2014

Related U.S. Application Data
(60) Provisional application No. 61/765,301, filed on Feb. 15, 2013.

(51) Int. Cl. C07D 207/16; C07F 7/18

(52) U.S. Cl. CPC C07D 207/16; C07F 7/18
USPC 514/63; 548/532; 514/423; 548/406

(57) ABSTRACT

The present invention relates to compounds of Formula (I), combinations and use thereof for disease therapy, or pharmaceutically acceptable salt or solvate thereof, including all tautomers, stereoismers and polymorphs thereof, which are iGluR receptor inhibitors, and hence are useful in the treatment of psychiatric diseases or neurological disorders or a disease or disorder associated with abnormal activities of iGluR receptors.

### FIELD OF THE INVENTION

The present invention relates to a class of substituted pyrrolidine-2-carboxylic acid derivatives as iGluR antagonist, their salt and solvates, pharmaceutical compositions comprising them, their use as medicament and in therapy, and preparation thereof. In particular, the invention relates to a class of substituted pyrrolidine-2-carboxylic acid derivatives as iGluR antagonist, which is useful in the treatment of psychiatric diseases or neurological disorders or a disease or disorder associated with abnormal activities of iGluR.

### BACKGROUND

In the mammalian central nervous system (CNS), (S)-glutamate (Glu) functions as the major excitatory neurotransmitter (FIG. 1).¹ The glutamatergic neurotransmitter system is involved in a vast number of basic neuro-physiological processes such as memory, cognition, as well as neuronal plasticity and development.²⁻⁹ Thus, psychiatric diseases or neurological disorders such as depression,¹⁰⁻¹² anxiety,¹³⁻¹⁵ addiction,¹⁶ migraine,¹⁷ and schizophrenia¹⁸⁻²² may be directly related to disordered glutamatergic neurotransmission. Moreover, elevated synaptic Glu levels or excessive Glu signaling is neurotoxic and will ultimately cause neuronal death.²³⁻²⁶ Thus, it is believed that neurodegenerative diseases such as Alzheimer's,²⁷⁻³¹ Huntington's,³² amyotrophic lateral sclerosis (ALS),³³ cerebral stroke,³⁴ and epilepsy³⁵ may indeed be the result of a malfunctioning glutamatergic neurotransmitter system which may be reversed by action of small molecule Glu ligands.¹

Once released from the pre-synaptic neuron into the synapse, Glu activates a number of pre- and post-synaptic Glu receptors. On the basis of the pharmacological profile and ligand selectivity the Glu receptors have been grouped in two main classes: the fast acting ligand gated ion channels named the ionotropic Glu receptors (iGluRs), which comprise the AMPA receptors (subunits GluA1-4), kainate (KA) receptors (subunits GluK1-5), and NMDA receptors (subunits GluN1, GluN2A-D and GluN3A-C),³⁶ The second main class is the G-protein coupled metabotropic Glu receptors (mGluRs, subunits mGluR1-8),³⁷ which produce a slower signal transduction through second messenger systems.

Functional NMDA receptors are tetrameric in structure, formed by the assembly of two heterodimers comprising one GluN1 subunit in combination with one of the GluN2A-D subunits.³⁶ The NMDA receptors are blocked (antagonized) by small-molecules acting as glycine antagonists, open channel blockers, non-competitive antagonists or competitive antagonists.³⁸ Within the latter class of NMDA blockers, small molecules such as D-AP5, (R)-CPP, CGS19755, UBP141 and NVP-AAM077³⁹ have been reported to antagonize the GluN2A-D subunits with varying degrees of subunit preference (2-10 fold).³⁸ Furthermore, peptides (e.g. Conantokin G) isolated from the venom of Conus geographus have shown to antagonize GluN1/GluN2B selectively over GluN1GluN2A (100 fold), but with only a 10-fold preference over GluN1/GluN2C and GluN1/2D.⁴⁰

FIG. 1. Chemical structures of (S)-Glu, NMDA and selected published competitive NMDA antagonists

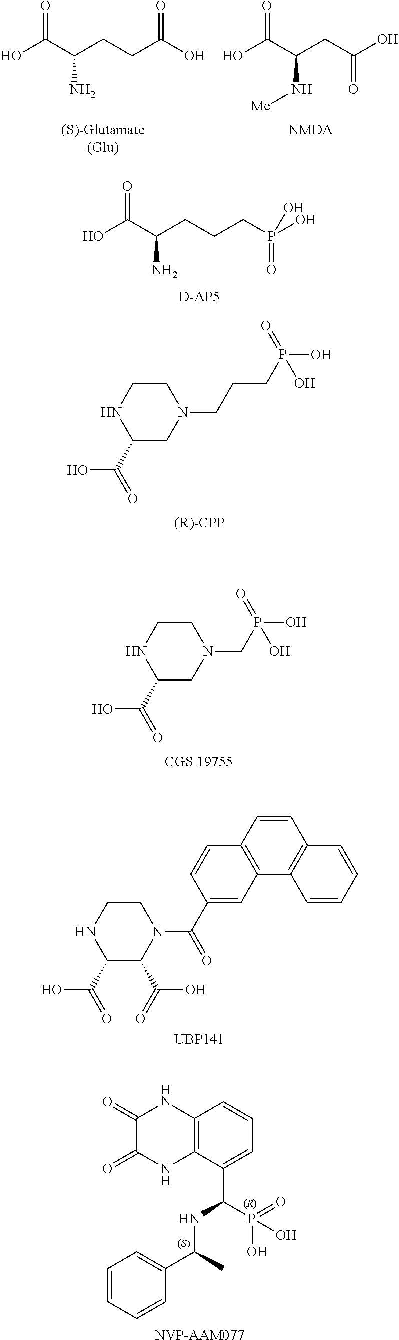

Further, disturbances of expression of KA receptors and function of KA receptors has been suggested to be linked to severe neurological- and psychiatric diseases.¹³⁸ Abnormal expression of KA subunit composition (GluK3 and GluK5) in the prefrontal cortex has been observed in schizophrenic subjects,¹³⁹ but also decreased expression of GluK2 and GluK3 from the medial dorsal thalamus to the dorsolateral prefrontal cortex and other cortical regions may be important to the pathophysiology of schizophrenia.¹⁴⁰ Furthermore, two population studies have suggested altered GluK3 expression (GRIK3 gene) as a risk factor,^{141,142} whereas GluK2 (GRIK2 gene) in one Japanese study came out short.¹⁴³ In bipolar disorder the GluK3 receptor is suggested to play a role,¹⁴⁴ but also intervention of GluK2 may constitute a therapeutic target.¹⁴⁵ In an rodent (rat) model of pain, trigeminal caudal nucleus nerve terminals mainly express GluK2/GluK3 subunits, which evidence that differentiated expression of KA receptor subtypes plays a role at the various stages of pain transmission.¹⁴⁶

To this date only selective antagonists for the GluK1 subtype has been reported.⁴⁷

FIG. 2 iGluR antagonist 1 (CNG-10100) and examples of selective high-affinity GluK1 antagonists LY466195⁴⁸ and UBP310.

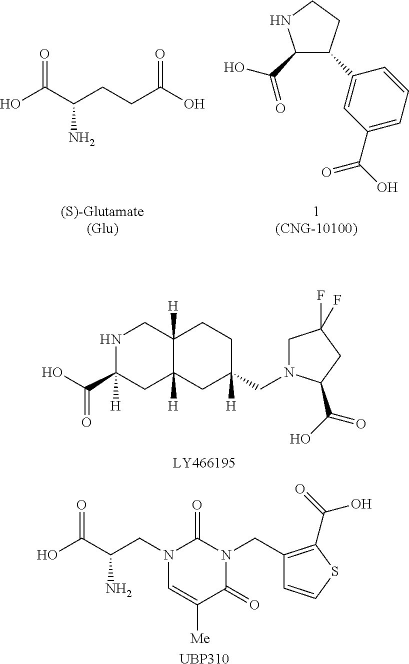

Hence, there is a strong need present for novel selective antagonists for iGluR or its subtypes such as GluA1-4, GluK1-5, or GluN1-3, such as GluN2A, GluN2B, GluN2C or GluN2D, which can be used to elucidate the role and function of iGluR receptors under both physiological and pathological conditions. Thus, new antagonists having high affinity and/or high specificity to one or more of the iGluR receptors such as GluA1-4, GluK1-5, or GluN1-3, such as GluN2A, GluN2B, GluN2C or GluN2D, would therefore be useful in the treatment of disorders and diseases associated with these receptors.

### SUMMARY OF THE INVENTION

With this background, it is an object of the present invention in a first embodiment to provide a compound of Formula (I)

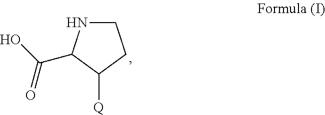

and pharmaceutically acceptable derivatives, as well as all tautomers and stereoisomers of compound of Formula (I), wherein

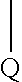

represents compounds of Formula (Ia) or (Ib);

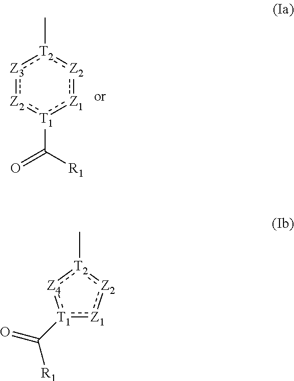

---- in each case may represent if appropriate the presence of at least one double bond between T₂ and Z₂, or between Z₂ and Z₁, or between Z₁ and T₁, or between T₁ and Z₄, or between Z₄ and (Z₃ or T₂), or between Z₃ and T₂; or

T₁ is C, CH,

T₂ is C, CH,

Z₁ is CR₂, C(R₂)₂, N, S, O, or NR₃,

Z₂ is CR₂, C(R₂)₂, N, S, O, or NR₃,

Z₃ is CR₂, C(R₂)₂, N, S, O, or NR₃,

Z₄ is CR₂, C(R₂)₂, N, S, O, or NR₃,

wherein the residues Z₁, Z₂, Z₃ and Z₄ can not represent adjacent O or S;

R₁ may together with Z₁ or Z₄, or

Z₂ may together with Z₁, or

Z₃ may together with Z₄,

form a saturated or unsaturated C₅- or C₆-cycloalkyl, or a saturated or unsaturated heterocyclyl containing 5 or 6 ring atoms, wherein the saturated or unsaturated C₅- or C₆-cycloalkyl, or the saturated or unsaturated heterocyclyl containing 5 or 6 ring atoms, may be substituted with one or more substituents selected from the group comprising OR₄ or R₄,

R₁ is H, OR₄, C₁-C₆-alkyl, C₂-C₆-alkenyl, C₂-C₆-alkynyl, COOR₄, N(OH)H, or NHR₄,

R₂ is independently selected among R₄, O, OR₄, halogen, N(OH)H, N(OH)R₄, NHR₄, COR₄, CONHR₄, or SO₂NHR₄,

R₃ is independently selected among R₄, O, OR₄, or halogen,

R₄ is independently selected among H, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine,

halogen represents Cl, Br, or I, and

with the proviso that Z₁, Z₂, Z₃ and Z₄ are not all CH, and T₁ and T₂ are not both C, when R₁ is OH.

In one particular embodiment of the present invention, the compounds of Formula (I) as previously described are stereoisomeric and contain at least two isomeric centers. Thus, depending on the orientation of the stereoisomers, presence of four different diastereomers is possible. Thus, compounds of Formula (I), wherein

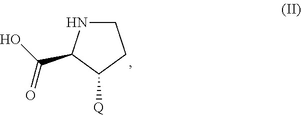

wherein

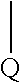

represents compounds of Formula (Ia1) or (Ib2);

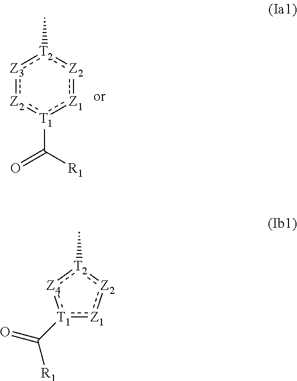

are also part of the invention.

In one particular embodiment of the present invention, Q represents a saturated ring.

Thus, compounds of Formula (I), wherein

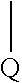

represents

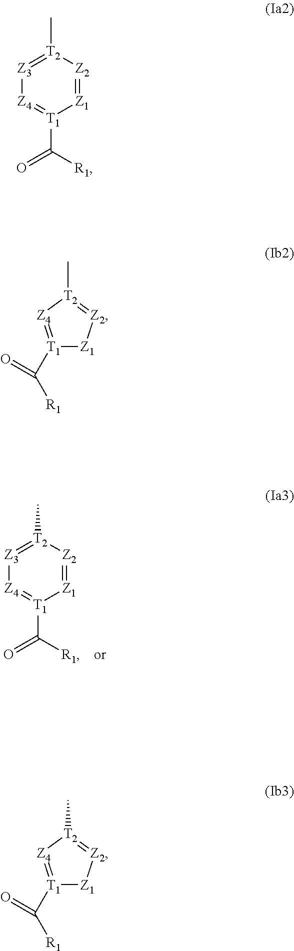

wherein

T₁ is C,

T₂ is C,

Z₁ is CR₂, N, S, O, or NR₃,

Z₂ is CR₂, or N,

Z₃ is CR₂, or N,

Z₄ is CR₂, or N,

wherein the residues Z₁, Z₂, and Z₃ cannot represent adjacent O or S;

R₁ may together with Z₁ or Z₄, or

Z₂ may together with Z₁ or Z₃,

form a saturated or unsaturated C₅- or C₆-cycloalkyl, or a saturated or unsaturated heterocyclyl containing 5 or 6 ring atoms, wherein the saturated or unsaturated C₅- or C₆-cycloalkyl, or the saturated or unsaturated heterocyclyl containing 5 or 6 ring atoms, may be substituted with one or more substituents selected from the group comprising OR₄ or R₄,

R₁ is H, OR₄, C₁-C₆-alkyl, C₂-C₆-alkenyl, C₂-C₆-alkynyl, COOR₄, N(OH)H, or NHR₄,

R₂ is R₄, O, OR₄, halogen, N(OH)H, N(OH)R₄, NHR₄, COR₄, CONHR₄, or SO₂NHR₄,

R₃ is R₄, O, OR₄, or halogen,

R₄ is H, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine,

halogen represents Cl, Br, or I

, are also part of the invention.

In a second embodiment of the present invention, the compound of Formula (I) as previously described can comprise additional ring forming structures wherein

R₁ may together with Z₁ or Z₄, or

Z₂ may together with Z₁, or

Z₃ may together with Z₄,

form a saturated or unsaturated C₅- or C₆-cycloalkyl, or a saturated or unsaturated heterocyclyl containing 5 or 6 ring atoms, wherein the saturated or unsaturated C₅- or C₆-cycloalkyl, or the saturated or unsaturated heterocyclyl containing 5 or 6 ring atoms, may be substituted with one or more substituents selected from the group comprising OR₄ or R₄.

In particular, in yet a further embodiment, the compound of Formula (I) as previously described is selected from the group consisting of

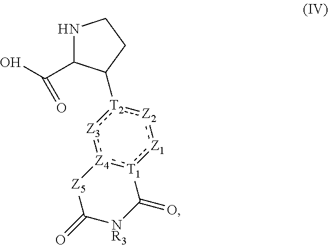

wherein

Z₅ is CR₂, C(R₂)₂, N, S, O, or NR₃,

wherein the residues Z₁, Z₂, Z₃, Z₄ and Z₅ cannot represent adjacent O or S,

----, T₁, T₂, Z₁, Z₂, Z₃, Z₄, R₂, R₃, R₄ have the same meaning as given above, or

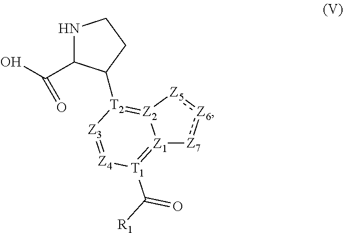

wherein

Z₅ is CR₂, C(R₂)₂, N, S, O, or NR₃,

Z₆ is CR₂, C(R₂)₂, N, S, O, or NR₃,

Z₇ is CR₂, C(R₂)₂, N, S, O, or NR₃,

wherein the residues Z₁, Z₂, Z₃, Z₄, Z₅, Z₆, Z₇ cannot represent adjacent O or S,

----, T₁, T₂, Z₁, Z₂, Z₃, Z₄, R₁, R₂, R₃, R₄ have the same meaning as given above.

In another embodiment of the present invention, the compound of Formula (I) as previously described is selected from the group consisting of

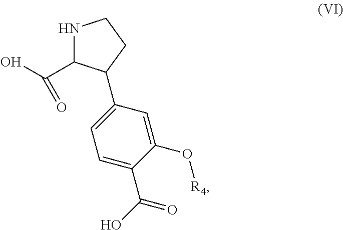

wherein

R₄ has the same meaning as given above,

are also part of the invention.

In one particular embodiment of the invention, compounds of Formula (I) are selected from the group wherein R₄ is alkyl, benzyl, alkylbiphenyl, preferably propyl, benzyl, or 3-methyl[1,1′-biphenyl].

Each of the described embodiments of the present invention is to be construed as disclosing the present invention either individually or in combination with the other embodiments.

### DETAILED DESCRIPTION OF THE INVENTION

In the following the present invention is described in more detail. All individual features and details can be individually applied to each embodiment and aspect of the compounds of Formula (I), its preparations, its formulations, its methods and its use.

The term “C₁₋₆ alkyl”, unless specifically limited, denotes a straight chain or branched alkyl group with 1, 2, 3, 4, 5 or 6 carbon atoms. Suitable C₁₋₆ alkyl groups include, for example, methyl, ethyl, propyl (e.g. n-propyl and isopropyl), butyl (e.g n-butyl, iso-butyl, sec-butyl and tert-butyl), pentyl (e.g. n-pentyl), and hexyl (e.g. n-hexyl).

The term “C₁₋₆ alkenyl”, unless specifically limited, may be interpreted similarly to the term “alkyl”. Alkenyl groups contain at least 1 double bond. Suitable alkenyl groups include ethenyl, propenyl, 1-butenyl, and 2-butenyl.

The term “C₁₋₆ alkynyl”, unless specifically limited, may be interpreted similarly to the term “alkyl”. Alkenyl groups contain at least 1 triple bond.

The term “saturated or unsaturated C₅- or C₆-cycloalkyl”, unless specifically limited, denotes cyclic carbon rings comprising 5 or 6 carbon atoms, wherein either a single or double bond between the mutually adjacent carbon atoms exist. Suitable saturated or unsaturated C₅- or C₆-cycloalkyl groups include cyclopentane, cyclohexane, cyclopentene, cyclohexene, cyclopenta-di-ene, cyclohhexa-di-ene, and phenyl.

The term “saturated or unsaturated heterocyclyl”, unless specifically limited, denotes a heterocyclic compound, such as a carbocyclyl group, phenyl group, or aryl residue, having atoms of at least two different elements as members of its ring. Suitable ring atoms in heterocyclic compound may be C, N, S, or O. Heterocyclic compounds according to the present invention may contain 3, 4, 5, 6, 7, 8 or even more rings atoms, preferably 5 or 6 ring atoms. Suitable saturated or unsaturated heterocyclic compounds may include pyrrolidine, pyrrole, tetrahydrofuran, furan, thiolane, thiophene, imidazolidine, pyrazolidine, imidazole, pyrazole, oxazolidine, isoxazolidine, oxazole, isoxazole, thiazolidine, isothiazolidine, thiazole, isothiazole, dioxolane, dithiolane, triazoles, furazan, oxadiazole, thiadiazole, dithiazole, tetrazole, piperidine, pyridine, oxane, pyran, thiane thiopyran, piperazine, diazines, morpholine, oxazine, thiomorpholine, thiazine, dioxane, dioxine, dithiane, dithiine, triazine, trioxane, or tetrazine.

The term “halogen” comprises fluorine (F), chlorine (Cl), bromine (Br) and iodine (I), more typically F, Cl or Br.

All possible tautomers of the claimed compounds are included in the present invention. Tautomers are isomers of organic compounds that readily interconvert by a chemical reaction called tautomerization. This reaction commonly results in the formal migration of a hydrogen atom or proton, accompanied by a switch of a single bond and adjacent double bond.

As compounds of Formula (I) contains at least 2 two asymmetric carbons, there are up to 4 possible configurations, which cannot all be non-superimposable mirror images of each other.

The compounds of the invention have one or more asymmetric centers. Compounds with asymmetric centers give rise to enantiomers (optical isomers), diastereomers (configurational isomers) or both, and it is intended that all of the possible enantiomers and diastereomers in mixtures and as pure or partially purified compounds are included within the scope of this invention. The present invention is meant to encompass all isomeric forms of the compounds of the invention. The present invention includes all stereoisomers of compounds of Formula (I). Compounds of Formula (I) comprises although depending on the choice of T₂ at least one chiral centers, i.e. at the second position of pyrrolidine, a COOH group, and at the third position of pyrrolidine, a 5- or 6-membered ring, as indicated by the Q-group. Diastereomers differ from enantiomers in that these are pairs of stereoisomers that differ in all stereocenters. Diastereomers have different physical properties (unlike enantiomers) and different chemical reactivity. Diastereoselectivity is the preference for the formation of one or more than one diastereomer over the other in an organic reaction.

The independent syntheses of the enantiomerically or diastereomerically enriched compounds, or their chromatographic separations, may be achieved as known in the art by appropriate modification of the methodology disclosed herein. Their absolute stereochemistry may be determined by the x-ray crystallography of crystalline products or crystalline intermediates that are derivatized, if necessary, with a reagent containing an asymmetric center of known absolute configuration. If desired, racemic mixtures of the compounds may be separated so that the individual enantiomers or diastereomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods using chiral stationary phases, which methods are well known in the art.

Alternatively, any enantiomer or diastereomer of a compound may be obtained by stereoselective synthesis using optically pure starting materials or reagents of known configuration by methods well known in the art.

Pyrrolidine-2-carboxylic acid derivatives according to the invention can be prepared from the various examples given further below or by consulting handbooks within organic chemistry. Examples of such handbook—although not intending to be limited thereto—are “Organic Chemistry, 2^{nd} Edition, 2000, by Maitland Jones, Jr., and Organic Chemistry, 6th Edition, Robert T. Morrison, and Robert N. Boyd. These two specifically referred handbooks are hereby incorporated by reference.

The term “pharmaceutically acceptable derivative” in present context includes pharmaceutically acceptable salts, which indicate a salt which is not harmful to the patient. Such salts include pharmaceutically acceptable basic or acid addition salts as well as pharmaceutically acceptable metal salts, ammonium salts and alkylated ammonium salts. A pharmaceutically acceptable derivative further includes hydrates, polymorphs, esters and prodrugs, or other precursors of a compound which may be biologically metabolized into the active compound, or crystal forms of a compound. Salts and solvates of the compounds of Formula (I) and physiologically functional derivatives thereof which are suitable for use in medicine are those wherein the counter-ion or associated solvent is pharmaceutically acceptable. However, salts and solvates having non-pharmaceutically acceptable counter-ions or associated solvents are within the scope of the present invention, for example, for use as intermediates in the preparation of other compounds and their pharmaceutically acceptable salts and solvates.

Pharmaceutically acceptable acid addition salts include those formed from hydrochloric, hydrobromic, sulfuric, nitric, citric, tartaric, phosphoric, lactic, pyruvic, acetic, trifluoroacetic, triphenylacetic, sulfamic, sulfanilic, succinic, oxalic, fumaric, maleic, malic, mandelic, glutamic, aspartic, oxaloacetic, methanesulfonic, ethanesulfonic, arylsulfonic (for example p-toluenesulfonic, benzenesulfonic, naphthalenesulfonic or naphthalenedisulfonic), salicylic, glutaric, gluconic, tricarballylic, cinnamic, substituted cinnamic (for example, phenyl, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, benzeneacrylic (for example 1,4-benzenediacrylic), isethionic acids, perchloric, propionic, glycolic, hydroxyethanesulfonic, pamoic, cyclohexanesulfamic, salicylic, saccharinic and trifluoroacetic acid.

Pharmaceutically acceptable base salts include ammonium salts, alkali metal salts such as those of sodium and potassium, alkaline earth metal salts such as those of calcium and magnesium and salts with organic bases such as dicyclohexylamine and [Lambda]/-methyl-D-glucamine.

Furthermore, some of the crystalline forms of the compounds may exist as polymorphs and as such are intended to be included in the present invention. In addition, some of the compounds may form solvates with water (i.e. hydrates) or common organic solvents, and such solvates are also intended to be encompassed within the scope of this invention. The compounds, including their salts, can also be obtained in the form of their hydrates, or include other solvents used for their crystallization.

Organic molecules can form crystals that incorporate water into the crystalline structure without modification of the organic molecule. An organic molecule can exist in different crystalline forms, each different crystalline forms may contain the same number of water molecules pr organic molecule or a different number of water molecules pr organic molecule.

The term “administering” shall encompass the treatment of the various disorders described with derivatives of the claimed compounds which convert to the active compound in vivo after administration to the subject. Conventional procedures for the selection and preparation of suitable prodrug derivatives are described, for example, in “Design of Prodrugs”, ed. H. Bundgaard, Elsevier, 1985.

The term “antagonist” in the present context refers to a substance that does not provoke a biological response itself upon binding to a receptor. Hence, antagonists have affinity to but no efficacy for their cognate receptors, and binding will disrupt the interaction and inhibit the function of e.g. an agonist.

The term “AMPA receptor” denotes a receptor family within iGluRs receptors. The AMPA receptor comprises of several subunits, such as GluA1, GluA2, GluA3, or GluA4.

The term “KA” denotes a receptor family within iGluRs receptors. The AMPA receptor comprises of several subunits, such as GluK1, GluK2, GluK3, GluK4, or GluK5.

The term “NMDA” denotes a receptor family within iGluRs receptors. The AMPA receptor comprises of several subunits, such as GluN1, GluN2A, GluN2B, GluN2C, GluN2D, GluN3A, GluN3B, or GluN3C.

A receptor antagonist defined by the Formula (I), is thus capable of binding to the GluK1, GluK2, GluK3, GluK4, or GluK5 receptor, respectively. The same considerations apply similarly to receptor antagonists of the AMPA receptor and NMDA receptor subunits.

The antagonist may be an antagonist of several different types of receptors, and thus capable of binding to several different types of receptors, such AMPA receptors, KA receptors, and NMDA receptors.

The antagonist can also be a selective antagonist which only binds to and activates one type of receptor. Antagonist may bind reversible or irreversible depending on the antagonist-receptor complex.

The term “IC₅₀” is commonly used as a measure of antagonist drug potency and reflects the measure of the effectiveness of a compound in inhibiting biological or biochemical function. This quantitative measure indicates how much of a compound of Formula (I) is needed to inhibit 50% of the activity of a particular receptor. IC₅₀ can be regarded as the functional strength of the different compounds of Formula (I).

IC₅₀ is not a direct indicator of affinity although the two can be related at least for competitive agonists and antagonists by the Cheng-Prusoff equation.

The term “kᵢ” refers to the binding affinity, which describe the binding of compounds of Formula (I) to a receptor.

As used herein, the term “pharmaceutical composition” is intended to encompass a product comprising compounds of Formula (I) in the therapeutically effective amounts, as well as any product which results, directly or indirectly, from combinations of the claimed compounds.

The term “therapeutically effective amount” of a compound as used herein refers to an amount sufficient to cure, alleviate, prevent, reduce the risk of, or partially arrest the clinical manifestations of a given disease or disorder and its complications. An amount adequate to accomplish this is defined as a “therapeutically effective amount”.

Compounds of Formula (I) according to the present invention may be used in pharmaceutical compositions and method for treatment of disorders, diseases in a subject, or conditions associated with the dysfunction of iGluR receptors, e.g. the AMPA receptors, KA receptors and NMDA receptors, and their corresponding subunits, GluA1-4, GluK1-5 and GluN1, GluN2A-D, and GluN3A-C. Thus, targeting iGluR or its subtypes such as GluA1-4, GluK1-5, GluN1-3 and its subtypes with antagonists according to the present invention would be helpful in the treatment of disorders and diseases associated with these receptors.

The terms “treatment” and “treating” as used herein refer to the management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” is to be understood to refer to the management and care of a patient for the purpose of hindering the development of the condition, disease or disorder, and includes the administration of the active compounds to prevent or reduce the risk of the onset of symptoms or complications.

The term “subject” refers to an animal, preferably a mammal, most preferably a human, who has been the object of treatment, observation or experiment. Treatment of animals, such as mice, rats, dogs, cats, cows, sheep and pigs, is, however, also within the scope of the present invention.

In a certain embodiment, the present invention relates to compounds of formula (I) or pharmaceutical compositions thereof, or methods for treatment of diseases or conditions binding one or more of the GluA1, GluA2, GluA3, GluA4, GluK1, GluK2, GluK3, GluK4, GluK5, GluN2A, GluN2B, GluN2C or GluN2D receptors subunits to obtain a beneficial therapeutic effect. Optionally, compounds of formula (I) or pharmaceutical compositions thereof are used in methods for treatment of diseases or conditions binding one or more of the GluA1, GluA2, GluA3, GluA4, GluK1, GluK2, GluK3, GluK4, GluK5, GluN2A, GluN2B, GluN2C or GluN2D receptors subunits to obtain a beneficial therapeutic effect

In yet a certain embodiment, the present invention relates to compounds of formula (I) or pharmaceutical compositions thereof, or methods for treatment of disorders of the central nervous system, neuro-physiological processes such as memory, cognition; as well as neuronal plasticity and development, psychiatric diseases or neurological disorders such as depression, anxiety, addiction, pain, migraine, and schizophrenia, and neurodegenerative diseases; such as Alzheimer, Huntington disease, amyotrophic lateral sclerosis (ALS), cerebral stroke, and epilepsy; and diseases including aching, ADHD, Autism, Diabetes, Huntington's disease, ischemia, multiple sclerosis, Parkinson's disease (Parkinsonism), Rasmussen's encephalitis, seizures, AIDS dementia complex, amyotrophic lateral sclerosis, combined systems disease (vitamin B12 deficiency), drug addiction, drug tolerance, drug dependency, glaucoma, hepatic encephalopathy, hydroxybutyric aminoaciduria, hyperhomocysteinemia and homocysteinuria, hyperprolinemia, lead encephalopathy, leber's disease, MELAS syndrome, MERRF, mitochondrial abnormalities (and other inherited or acquired biochemical disorders), neuropathic pain syndromes (e.g. causalgia or painful peripheral neuropathies), nonketotic hyperglycinemia, olivopontocerebellar atrophy, essential tremor, Rett syndrome, sulfite oxidase deficiency, Wernicke's encephalopathy or cancer.

According to the present invention, the antagonist of compound of formula (I) is administered to subjects in need of treatment in pharmaceutically effective doses. A therapeutically effective amount of a compound according to the present invention is an amount sufficient to cure, prevent, reduce the risk of, alleviate or partially arrest the clinical manifestations of a given disease or its complications. The amount that is effective for a particular therapeutic purpose will depend on the severity and the sort of the disease as well as on the weight and general state of the subject. The antagonists of the present invention may be administered one or several times per day, such as from 1 to 4 times per day, such as from 1 to 3 times per day, such as from 1 to 2 times per day, wherein administration from 1 to 3 times per day is preferred.

In a preferred embodiment, the present invention relates to antagonist of compound of formula (I) which is administered in doses of 0.5-1500 mg/day, preferably 0.5-200 mg/day, more preferably 0.5-60 mg/day, even more preferably 0.5-30 mg/day.

In a preferred embodiment, the present invention relates to antagonist of compound of formula (I) suitable for oral, rectal, nasal, pulmonary, buccal, sublingual, transdermal or parenteral administration.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991, fully incorporated herein by reference. The protecting groups may be removed at a convenient subsequent stage using methods known from the art.

In a preferred embodiment, the present invention relates to compounds of formula (I), such as
(1) (2S,3R)-3-(4-Carboxyphenyl)pyrrolidine-2-carboxylic Acid
(2) (2S,3R)-tert-Butyl 2-(((tert-butyldimethylsilyl)oxy)methyl)-3-(4-(((tert-butyldimethylsilyl)oxy)-methyl)phenyl)-5-oxopyrrolidine-1-carboxylate
(3) (2S,3R)-tert-Butyl 2-(((tert-butyldimethylsilyl)oxy)methyl)-3-(4-(((tert-butyldimethylsilyl)oxy)-methyl)phenyl)pyrrolidine-1-carboxylate
(4) (2S,3R)-tert-Butyl 2-(hydroxymethyl)-3-(4-(hydroxymethyl)phenyl)pyrrolidine-1-carboxylate
(5) (2S,3R)-3-(3-(Benzyloxy)-5-carboxyphenyl)-1-(tert-butoxycarbonyl)pyrrolidine-2-carboxylic acid

### EXAMPLES

### Results and Discussion

Chemistry

The synthesis of 2 followed the following general strategy which also is possible for 1. Commercially available optically pure (S)-glutaminol (3), was firstly double protected to give 4, after which a double bond was introduces under standard selenylation/oxidative conditions to give enone 5. TBS protection of commercially available 4-bromobenzylalcohol 11 gave phenylbromide 6 in quantitative yield (Scheme 6). Copper catalyzed conjugate addition of 6 to enone 5 gave the desired product 7 as a single diastereomer in high yield (84%). Reduction of the endo-cyclic carbonyl group by borane gave pyrrolidinone 8 in acceptable yield (59%). After removal of the TBS groups by treatment with TBAF, the diol 9 was oxidized using a ruthenium catalyzed methodology to give the desired diacid 10 in high yield (86%). Finally, removal of the BOC group by TFA in DCM gave the target compound 2 in (74%).

FIG. 3. Chemical structures designed iGluR antagonist 1 (CNG-10100) and repositioning of the distal carboxylic acid group to give analog 2 (CNG-10101).

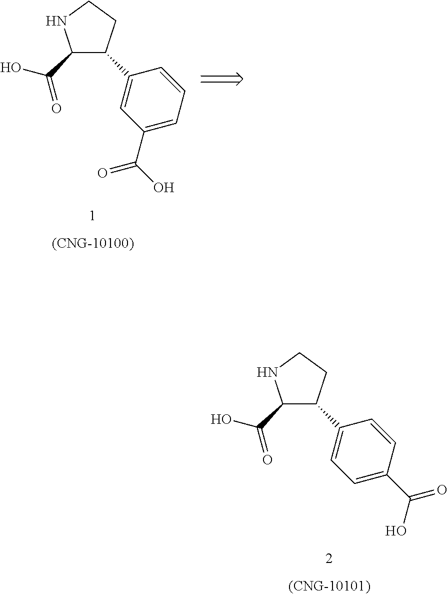 [SMILES: O=C(O)c1cccc([C@H]2CCN[C@@H]2C(=O)O)c1 ; O=C(O)c1ccc([C@H]2CCN[C@@H]2C(=O)O)cc1]

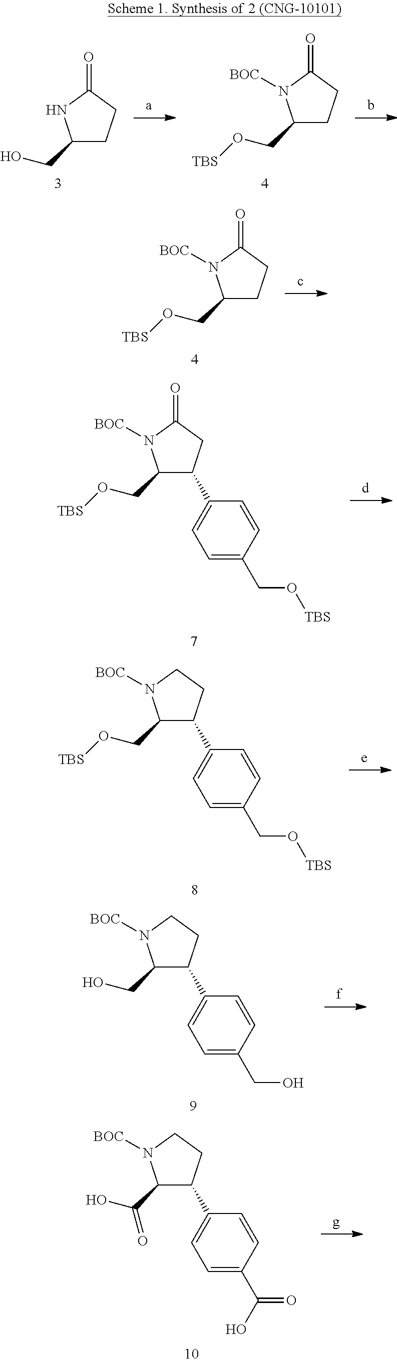

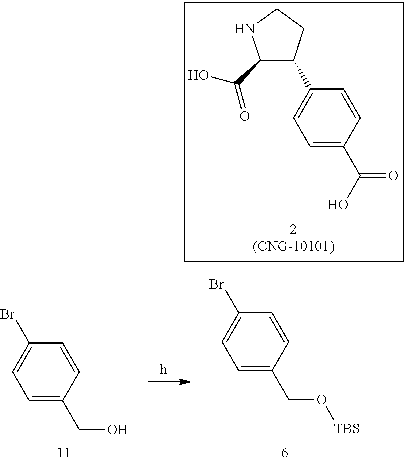

Reagents and conditions. a) TBSCl, imidazole, DCM, rt, 24 h, aq workup, then BOC₂O, DMAP, ACN, rt, 16 h (two steps, 91%); b) LHMDS, PhSeCl, THF, −78° C., then 30% H₂O₂, EtOAc, 0° C. to rt (two steps, 64%); c) 6f, tert-BuLi, CuCN, Et₂O, −78 to −42° C. (84%); d) BH₃, THF, reflux, 31/2h, then H₂O, NaOH, H₂O₂, rt, 3½ h (59%); e) TBAF, THF, rt, 18 h (88%); f) NaIO₄, RuCl₃, 0° C., 1½ h (86%); g) TFA:DCM, rt, 18 h (74%). h) TBSCl, imidazole, DMF, 0° C. to rt, 1½ h (quant.);

Pharmacological Characterization

Analog 2 was characterized pharmacologically in radio ligand binding assays at native iGluRs (rat synaptosomes) and cloned homomeric subtypes, GluK1-3 (Table 1). At native iGluRs 2 showed neglectable affinity for AMPA receptors and KA receptors (>100 μM) but most interestingly, micro molar affinity for the NMDA receptors (IC₅₀=8.5 μM). At cloned homomeric GluK1-3 receptors 2 displayed no affinity (>100 μM).

TABLE 1
Chemical structures and pharmacological characterization
of 1 and 2 at native iGluR receptors as well as cloned
homomeric GluK1-3 subtypes. All values in μM

| | AMPA | KA | NMDA | GluK1 | GluK2 | GluK3 |
|---|---|---|---|---|---|---|
| | IC₅₀ | IC₅₀ | Kᵢ | Kᵢ | Kᵢ | Kᵢ |
| 1^{a} | 51 | 22 | 6.0 | 4.3 | >100 | 8.1 |
| (CNG- | | | | | | |
| 10100) | | | | | | |
| 2 | >100 | >100 | 8.5 | >100 | >100 | >100 |
| (CNG- | | | [5.07 ± 0.04] | | | |
| 10101) | | | | | | |

^{a}Values taken from reference 49.

Pharmacological characterization in a functional assay for subtypes GluN1/GluN2A-D revealed that 2 was indeed an antagonist.

All reagents were obtained from commercial suppliers and used without further purification. Dry solvents were obtained differently. THF was distilled over sodium/benzophenone. Et₂O was dried over neatly cut sodium. All solvents were tested for water content using a Carl Fisher apparatus. Water—or air sensitive reactions were conducted in flame dried glassware under nitrogen with syringe-septum cap technique. Purification by DCVC (dry column vacuum chromatography) was performed with silica gel size 25-40 μm (Merck, Silica gel 60). For TLC was used Merck TLC Silica gel F₂₅₄ with appropriate spray reagents: KMnO₄ or Molybdenum blue. ¹H NMR and ¹³C NMR spectra were obtained on a Varian Mercury Plus (300 MHz) and a Varian Gemini 2000 instrument (75 MHz), respectively. HPLC was done using Agilent Prep HPLC systems with Agilent 1100 series pump, Agilent 1200 series diode array, multiple wavelength detector (G1365B), and Agilent PrepHT High Performance Preparative Cartridge Column (Zorbax, 300 SB-C18 Prep HT, 21.2×250 mm, 7 μm). Preparative HPLC was performed using Spectraseries UV100 with a JASCO 880-PU HPLC pump and an XTerra® Prep MS C18, (10 μm, 10×300 mm) column. LC-MS was performed using an Agilent 1200 series solvent delivery system equipped with an autoinjector coupled to an Agilent 6400 series triple quadrupole mass spectrometer equipped with an electrospray ionization source. Gradients of 10% aqueous acetonitrile+0.05% formic acid (buffer A) and 90% aqueous acetonitrile+0.046% formic acid (buffer B) were employed or an Agilent 1200 system using a C18 reverse phase column (Zorbax 300 SB-C18, 21.1 mm-250 mm) with a linear gradient of the binary solvent system of H₂O/CH₃CN/TFA (A: 100/0/0.1 and B: 5/95/0.1) with a flow rate of 20 mL/min. Optical rotation was measured using a Perkin-Elmer 241 spectrometer, with Na lamp at 589 nm. Melting points were measured using an automated melting point apparatus, MPA100 OptiMelt (SRS) and are stated uncorrected. Compounds were dry either under high vacuum or freeze dried using a Holm & Halby, Heto LyoPro 6000 freezedrier.

### (2S,3R)-3-(4-Carboxyphenyl)pyrrolidine-2-carboxylic Acid (2f)

Diacid 10f (215 mg, 0.64 mmol, 1.00 equiv) was dissolved in DCM (5 mL) at rt and TFA (4 mL) was added. The mixture was stirred for 18 h at rt under argon. The mixture was concentrated in vacuo to yield a clear, colorless foam. The crude product was dissolved in H₂O (4 mL) and a white, amorph solid precipitated. The solid was dried to yield 112 mg (74%) of 2f as the twitterion. ¹H NMR: (300 MHz, NaOD in D₂O): δ 1.48 (1H, m), 1.80 (1H, m), 2.59 (1H, m), 2.67-2.80 (2H, m), 3.00 (1H, d, J=9 Hz), 6.94 (2H, d, J=8 Hz), 7.39 (2H, d, J=8 Hz); ¹³C NMR: (75 MHz, NaOD in D₂O): δ 36.0, 46.9, 51.7, 70.7, 128.1, 130.0, 135.0, 147.3, 176.1, 181.7; LCMS: m/z [M+H]⁺: calc: 236.08. found: 236.0; OR: [a]²²_{D}: +21.97 (c=0.47 g/100 mL; 2M NaOH).

### (2S,3R)-tert-Butyl 2-(((tert-butyldimethylsilyl)oxy)methyl)-3-(4-(((tert-butyldimethylsilyl)oxy)-methyl)phenyl)-5-oxopyrrolidine-1-carboxylate (7f)

Solution A: Thiophen (487 mg, 5.79 mmol, 1.90 equiv) was dissolved in dry Et₂O (8 mL) in a dry vial under N₂. The mixture was cooled to 0° C. and dropwise added 2.03 M n-BuLi (2.85 mL, 5.79 mmol, 1.90 equiv) over the course of 15 min. The mixture was left to stir at 0° C. for 15 min. before removed from the ice bath and stirred at r.t. for 1 h (a white, colloid precipitate was formed).

Bromide 6f (1.15 g, 3.82 mmol, 1.25 equiv) was dissolved in dry Et₂O (30 mL) in a dry flask under N₂. The mixture was cooled to −78° C. and dropwise added 1.55 M tert-BuLi (4.95 mL, 7.67 mmol, 2.51 equiv) over the course of 20 min. The mixture was left to stir at −78° C. for 15 min. (The solution became yellow/braunish colored and unclear) before a suspension of CuCN (342 mg, 3.82 mmol, 1.25 equiv) in dry Et₂O (5 mL) was added dropwise over the course of 5 min. The mixture was left to stir at −42° C. for 10 min (CuCN dissolves and gives clear solution) then recooled to −78° C. To the mixture was dropwise added Solution A (7.5 mL, 3.8 mmol, 1.3 equiv) over the course of 15 min. and left to stir at −78° C. for 10 min, then at −42° C. for 10 min before recooled to −78° C. A solution of 6f (1.00 g, 3.05 mmol, 1.00 equiv) dissolved in Et₂O (4 mL) was added dropwise over 20 min. (color change to deep yellow). The mixture was left to stir at −78° C. for 15 min., then at −42° C. for 60 min before quenched with sat. NaHCO₃ (6 mL). The mixture was transferred to a separation funnel containing sat. NaHCO₃ (75 mL) and EtOAc (40 mL). The aqueous phase was extracted with EtOAc (2×50 mL) and the combined organic layers washed with brine (75 mL), dried over MgSO₄, filtered and concentrated in vacuo. The crude product was purified by DCVC (diameter=7 cm, 75 mL fractions, 0-4% EtOAc in heptanes) to yield 1.41 g (84%) of 7f as a clear, colorless oil, which solidified upon standing. ¹H NMR (300 MHz, CDCl₃): δ 0.08 (3H, s), 0.10 (3H, s), 0.12 (6H, s), 0.92 (9H, s), 0.96 (9H, s), 1.54 (9H, s), 2.53 (1H, dd, J=18, 3 Hz), 3.15 (1H, dd, J=18, 10 Hz), 3.45 (1H, dm, J=9 Hz), 3.80 (1H, dd, J=11, 2 Hz), 4.00 (1H, dd, J=11, 4 Hz), 4.06 (1H, m), 4.72 (2H, s), 7.15 (2H, d, J=8 Hz), 7.28 (2H, d, J=8 Hz); ¹³C NMR (75 MHz, CDCl₃): δ −5.2, −5.0, 18.4, 18.7, 26.1, 26.2, 28.3, 38.7, 40.2, 63.8, 64.7, 67.0, 83.1, 126.3, 126.8, 140.3, 142.9, 149.8, 174.2 LCMS: m/z [M+H]⁺: calc: 450.3. found: 450.3; TLC: R_{f}=0.41 (EtOAc:heptanes (1:4)); OR: [a]²²_{D}: −27.55 (c=0.65 g/100 mL; EtOAc).

### (2S,3R)-tert-Butyl 2-(((tert-butyldimethylsilyl)oxy)methyl)-3-(4-(((tert-butyldimethylsilyl)oxy)-methyl)phenyl)pyrrolidine-1-carboxylate (8f)

Compound 7f (1.43 g, 2.35 mmol, 1.00 equiv) was dissolved in dry THF (15 mL) and added 1M BH₃.THF complex (30.0 mL, 30.0 mmol, 11.7 equiv) over the course of 5 min. The mixture was refluxed under N₂ for 18 h. The mixture was cooled to 0° C. and dropwise added H₂O (6 mL) over the course of 2 h, NaOH (2M, 30 mL) dropwise over the course of 40 min and H₂O₂ (30%, 10 mL) over the course of 15 min (organic/aqueous ratio important). After 5 min the mixture was remove from the ice bath and left to stir at r.t. for 1.5 h. The mixture was poured into a separating funnel containing sat. NaHCO₃ (100 mL) and EtOAc (50 mL). After separation the aqueous phase was extracted with EtOAc (2×75 mL) and the combined organic layers washed with brine (100 mL), dried over MgSO₄, filtered, and concentrated in vacuo. The crude product was purified by DCVC (diameter=4 cm, 30 mL fractions, 0-4% EtOAc in toluene) to yield 8f (809 mg, 59%) as a clear, colorless oil. ¹H NMR (300 MHz, CDCl₃): δ 0.06 (6H, s), 0.13 (6H, s), 0.92 (9H, s), 0.97 (9H, s), 1.23-1.38 (1H, m), 1.50 (9H, s), 1.83-1.98 (1H, m), 2.18-2.34 (1H, m), 3.25-3.45 (1H, m), 3.47-3.80 (4H, m), 3.85 (0.53H, m), 4.04 (0.41H, m), 4.73 (2H, s), 7.1-7.23 (2H, m), 7.23-7.30 (2H, m); ¹³C NMR (75 MHz, CDCl₃): δ −5.1, −5.0, 18.4, 18.7, 26.1, 26.2, 28.8, 31.8, 33.0, 45.4, 46.3, 46.5, 47.2, 61.4, 62.9, 64.9, 65.6, 65.8, 79.1, 79.5, 126.4, 127.1, 127.3, 139.6, 142.1, 142.7, 154.2, 154.3; LCMS: m/z [M+H]⁺: calc: 436.3. found: 436.3; TLC: R_{f}=0.54 (EtOAc:toluene (1:6)); OR: [a]²²_{D}: +7.27 (c=0.87 g/100 mL; EtOAc).

### (2S,3R)-tert-Butyl 2-(hydroxymethyl)-3-(4-(hydroxymethyl)phenyl)pyrrolidine-1-carboxylate (9f)

Compound 8f (722 mg, 1.34 mmol, 1.00 equiv) was dissolved in dry THF (10 mL) and added 1M TBAF (5 mL). The mixture was left to stir under nitrogen for 18 h. The mixture was transferred to a separation funnel containing EtOAc (50 mL) and sat. NaHCO₃ (50 mL). The aqueous phase was reextracted with EtOAc (2×50 mL). The pooled organic phases was washed with brine (75 mL), dried over MgSO₄, filtered and concentrated in vacuo. The crude product was purified by DCVC (diameter=3 cm, 30 mL fractions, 0-100% EtOAc in heptanes) to yield 9f (364 mg, 88%) as a clear, colorless oil. ¹H NMR (300 MHz, CDCl₃): δ 1.48 (9H, s), 1.91 (1H, m), 2.12 (1H, m), 2.91 (1H, m), 3.20-3.45 (2H, m), 3.51-3.80 (3H, m), 3.88 (1H, m), 4.57 (2H, s), 5.20 (1H, m), 7.15 (2H, d, J=8 Hz), 7.25 (2H, d, J=8 Hz); ¹³C NMR (75 MHz, CDCl₃): δ 28.6, 32.9, 47.1, 47.4, 64.5, 65.5, 66.8, 80.6, 127.3, 127.5, 139.8, 140.0, 156.6; LCMS: m/z [M+H]⁺: calc: 252.1. found: 252.1 (decomposition of tert-Bu group); TLC: R_{f}=0.37 (EtOAc:heptane (3:1)); OR: [a]²²_{D}: +3.98 (c=0.43 g/100 mL; EtOAc).

### (2S,3R)-3-(3-(Benzyloxy)-5-carboxyphenyl)-1-(tert-butoxycarbonyl)pyrrolidine-2-carboxylic acid (10f)

Suspension A: NaIO₄ (1.92 g, 8.98 mmol, 9.87 equiv) and RuCl₃. H₂O (8 mg, 0.037 mmol, 0.04 equiv) were suspended in H₂O (6 mL) and stirred at r.t. for 1 min. prior to use.

Diol 9f (280 mg, 0.91 mmol, 1.00 equiv) was dissolved in CH₃CN (5 mL) and EtOAc (5 mL), cooled to 0° C. and dropwise added suspension A over the course of 15 min. The flask containing suspension A was washed with H₂O (2 mL), which was added the mixture over the course of 5 min. The mixture was left to stir at 0° C. for 1.5 h. The mixture was filtered into a separation funnel through a plug of celite, which was afterwards washed with water (20 mL) and EtOAc (2×20 mL). The pH of the aqueous phase was adjusted (pH˜2) by addition a few of drops of 2 M HCl. Sat. NaCl (5 mL) was added for complete separation of the phases. After separation the aqueous phase was extracted with EtOAc (2×20 mL) and the pooled organic phases were washed with brine (50 mL), dried over MgSO₄, filtered, and concentrated in vacuo. The crude product was purified by DCVC (diameter=3 cm, 25 mL fractions, 0-50% EtOAc in heptanes containing 2% AcOH) to yield 263 mg (86%) of 10f as a white foam. ¹H NMR (300 MHz, DMSO): δ 1.35 (6H, s), 1.42 (3H, s), 2.00 (1H, m), 2.21 (1H, m), 3.33-3.50 (2H, m), 3.50-3.62 (1H, m), 4.06 (0.66H, d, J=8 Hz), 4.09 (0.33H, d, J=7 Hz), 7.41 (0.66H, d, J=8 Hz), 7.43 (1.33H, d, J=8 Hz), 7.88 (2H, d, J=8 Hz); ¹³C NMR (75 MHz, DMSO): δ 28.0, 28.2, 32.1, 32.7, 46.0, 46.1, 48.2, 49.3, 65.1, 65.4, 79.1, 127.3, 127.5, 129.4, 129.5, 145.8, 146.1, 152.7, 153.2, 166.9, 172.8, 173.3; LCMS: m/z [M+H]⁺: calc: 236.1. found: 236.1 (-Boc); TLC: R_{f}=0.27 (EtOAc:heptanes:AcOH (40:20:1)); OR: [a]²²_{D}: +81.90 (c=0.33 g/100 mL; EtOAc).

### REFERENCES

(1) Meldrum, B. S. Glutamate as a Neurotransmitter in the Brain: Review of Physiology and Pathology. J. Nutr. 2000, 130, 1007-1007.
(2) Bleakman, D.; Lodge, D. Neuropharmacology of AMPA and kainate receptors. Neuropharmacology 1998, 37, 1187-1204.
(3) Chittajallu, R.; Braithwaite, S. P.; Clarke, V. R. J.; Henley, J. M. Kainate receptors: subunits, synaptic localization and function. Trends in Pharmacological Sciences 1999, 20, 26-35.
(4) Lerma, J.; Paternain, A. V.; Rodriguez-Moreno, A.; Lopez-Garcia, J. C. Molecular physiology of kainate receptors. Physiological Reviews 2001, 81, 971-998.
(5) Rozas, J. L.; Paternain, A. V.; Lerma, J. Noncanonical signaling by ionotropic kainate receptors. Neuron 2003, 39, 543-53.
(6) Pinheiro, P.; Mulle, C. Kainate receptors. Cell and Tissue Research 2006, 326, 457-482.
(7) Lovinger, D. M. Neurotransmitter roles in synaptic modulation, plasticity and learning in the dorsal striatum. Neuropharmacology 2010, 58, 951-961.
(8) Anggono, V.; Huganir, R. L. Regulation of AMPA receptor trafficking and synaptic plasticity. Current Opinion in Neurobiology 2012, 22, 461-469.
(9) Kalivas, P. W. Cocaine and amphetamine-like psychostimulants: neurocircuitry and glutamate neuroplasticity. Dialogues in clinical neuroscience 2007, 9, 389-97.
(10) McCarthy, D. J.; Alexander, R.; Smith, M. A.; Pathak, S.; Kanes, S.; Lee, C.-M.; Sanacora, G. Glutamate-based depression GBD. Medical Hypotheses 2012, 78, 675-681.
(11) Sanacora, G.; Treccani, G.; Popoli, M. Towards a glutamate hypothesis of depression: An emerging frontier of neuropsychopharmacology for mood disorders. Neuropharmacology 2012, 62, 63-77.
(12) Chaki, S.; Okubo, T.; Sekiguchi, Y. Non-monoamine-based approach for the treatment of depression and anxiety disorders. Recent Patents CNS Drug Discov. 2006, 1, 1-27.
(13) Harvey, B. H.; Shahid, M. Metabotropic and ionotropic glutamate receptors as neurobiological targets in anxiety and stress-related disorders: focus on pharmacology and preclinical translational models. Pharmacol Biochem Behav 2012, 100, 775-800.
(14) Riaza Bermudo-Soriano, C.; Perez-Rodriguez, M. M.; Vaquero-Lorenzo, C.; Baca-Garcia, E. New perspectives in glutamate and anxiety. Pharmacology Biochemistry and Behavior 2012, 100, 752-774.
(15) Michelson, D.; Levine, L. R.; Dellva, M. A.; Mesters, P.; Schoepp, D. D.; Dunayevich, E.; Tollefson, G. D. Clinical studies with mGluR2/3 agonists: LY354740 compared with placebo in patients with generalized anxiety disorder. Neuropharmacology 2005, 49, 257-257.
(16) Lea, P. M. th; Faden, A. I. Metabotropic glutamate receptor subtype 5 antagonists MPEP and MTEP. CNS drug reviews 2006, 12, 149-66.
(17) Vikelis, M.; Mitsikostas, D. D. The role of glutamate and its receptors in migraine. CNS Neurol. Disord. Drug Targets 2007, 6, 251-7.
(18) Javitt, D. C. Glutamatergic theories of schizophrenia. Isr J Psychiatry Relat Sci 2010, 47, 4-16.
(19) Javitt, D. C. Glutamate and schizophrenia: phencyclidine, N-methyl-D-aspartate receptors, and dopamine-glutamate interactions. Int. Rev. Neurobiol. 2007, 78, 69-108.
(20) Fell, M. J.; McKinzie, D. L.; Monn, J. A.; Svensson, K. A. Group II metabotropic glutamate receptor agonists and positive allosteric modulators as novel treatments for schizophrenia. Neuropharmacology 2012, 62, 1473-1483.
(21) Rotaru, D. C.; Lewis, D. A.; Gonzalez-Burgos, G. The role of glutamatergic inputs onto parvalbumin-positive interneurons: relevance for schizophrenia. Rev Neurosci 2012, 23, 97-109.
(22) Coyle, J. T.; Basu, A.; Benneyworth, M.; Balu, D.; Konopaske, G. Glutamatergic synaptic dysregulation in schizophrenia: therapeutic implications. Handb Exp Pharmacol 2012, 267-295.
(23) Almeida, A.; Heales, S. J.; Bolanos, J. P.; Medina, J. M. Glutamate neurotoxicity is associated with nitric oxide-mediated mitochondrial dysfunction and glutathione depletion. Brain research 1998, 790, 209-16.
(24) Gu, B.; Nakamichi, N.; Zhang, W.-S.; Nakamura, Y.; Kambe, Y.; Fukumori, R.; Takuma, K.; Yamada, K.; Takarada, T.; Taniura, H.; Yoneda, Y. Possible protection by notoginsenoside R1 against glutamate neurotoxicity mediated by N-methyl-D-aspartate receptors composed of an NR1/NR2B subunit assembly. J. Neurosci. Res. 2009, 87, 2145-2156.
(25) Kumar, A.; Singh, R. L.; Babu, G. N. Cell death mechanisms in the early stages of acute glutamate neurotoxicity. Neurosci. Res. 2010, 66, 271-278.
(26) Miao, Y.; Dong, L.-D.; Chen, J.; Hu, X.-C.; Yang, X.-L.; Wang, Z. Involvement of calpain/p35-p25/Cdk5/NMDAR signaling pathway in glutamate-induced neurotoxicity in cultured rat retinal neurons. PLoS ONE 2012, 7, e42318.
(27) Danysz, W.; Parsons, C. G. Alzheimer's disease, β-amyloid, glutamate, NMDA receptors and memantine--searching for the connections. Br. J. Pharmacol. 2012, 167, 324-352.
(28) McKeage, K. Memantine: a review of its use in moderate to severe Alzheimer's disease. CNS Drugs 2009, 23, 881-97.
(29) Kavirajan, H. Memantine: a comprehensive review of safety and efficacy. Expert opinion on drug safety 2009, 8, 89-109.
(30) Francis, P. T. Glutamatergic approaches to the treatment of cognitive and behavioural symptoms of Alzheimer's disease. Neurodegener Dis. 2008, 5, 241-3.
(31) Hu, N.-W.; Ondrejcak, T.; Rowan, M. J. Glutamate receptors in preclinical research on Alzheimer's disease: update on recent advances. Pharmacol. Biochem. Behay. 2012, 100, 855-862.
(32) Fan, M. M.; Raymond, L. A. N-methyl-D-aspartate (NMDA) receptor function and excitotoxicity in Huntington's disease. Progress in neurobiology 2007, 81, 272-93.
(33) Corona, J. C.; Tovar-y-Romo, L. B.; Tapia, R. Glutamate excitotoxicity and therapeutic targets for amyotrophic lateral sclerosis. Expert opinion on therapeutic targets 2007, 11, 1415-28.
(34) Muir, K. W. Glutamate-based therapeutic approaches: clinical trials with NMDA antagonists. Current Opinion In Pharmacology 2006, 6, 53-60.
(35) Alexander, G. M.; Godwin, D. W. Metabotropic glutamate receptors as a strategic target for the treatment of epilepsy. Epilepsy Research 2006, 71, 1-22.
(36) Traynelis, S. F.; Wollmuth, L. P.; McBain, C. J.; Menniti, F. S.; Vance, K. M.; Ogden, K. K.; Hansen, K. B.; Yuan, H.; Myers, S. J.; Dingledine, R. Glutamate receptor ion channels: structure, regulation, and function. Pharmacol Rev 2010, 62, 405-496.
(37) Vaidyaa, A.; Jainb, S.; Jaina, A. K.; Agrawala, A.; Kashawa, S. K.; Jaina, S. K.; Agrawala, R. K. Metabotropic Glutamate Receptors: A Review on Prospectives and Therapeutic Aspects. Mini Rev Med Chem 2012.
(38) Ogden, K. K.; Traynelis, S. F. New advances in NMDA receptor pharmacology. Trends in Pharmacological Sciences 2011, 32, 726-733.
(39) Frizelle, P. A.; Chen, P. E.; Wyllie, D. J. A. Equilibrium Constants for (R)-[(S)-1-(4-Bromo-phenyl)-ethylamino]-(2,3-dioxo-1,2,3,4-tetrahydroquinoxalin-5-yl)-methyl]-phosphonic Acid (NVP-AAM077) Acting at Recombinant NR1/NR2A and NR1/NR2B N-Methyl-d-aspartate Receptors: Implications for Studies of Synaptic Transmission. Mol Pharmacol 2006, 70, 1022-1032.
(40) Teichert, R. W.; Jimenez, E. C.; Twede, V.; Watkins, M.; Hollmann, M.; Bulaj, G.; Olivera, B. M. Novel conantokins from Conus parius venom are specific antagonists of N-methyl-D-aspartate receptors. J. Biol. Chem. 2007, 282, 36905-36913.
(138) Jane, D. E.; Lodge, D.; Collingridge, G. L. Kainate receptors: Pharmacology, function and therapeutic potential. Neuropharmacology 2009, 56, 90-113.
(139) Meador-Woodruff, J. H.; Davis, K. L.; Haroutunian, V. Abnormal kainate receptor expression in prefrontal cortex in schizophrenia. Neuropsychopharmacology 2001, 24, 545-552.
(140) Sodhi, M. S.; Simmons, M.; McCullumsmith, R.; Haroutunian, V.; Meador-Woodruff, J. H. Glutamatergic gene expression is specifically reduced in thalamocortical projecting relay neurons in schizophrenia. Biol Psychiatry 2011, 70, 646-54.
(141) Ahmad, Y.; Bhatia, M. S.; Mediratta, P. K.; Sharma, K. K.; Negi, H.; Chosdol, K.; Sinha, S. Association between the ionotropic glutamate receptor kainate3 (GRIK3) Ser310Ala polymorphism and schizophrenia in the Indian population. World J Biol Psychiatry 2009, 10, 330-3.
(142) Djurovic, S.; Kahler, A. K.; Kulle, B.; Jonsson, E. G.; Agartz, I.; Le Hellard, S.; Hall, H.; Jakobsen, K. D.; Hansen, T.; Melle, I.; Werge, T.; Steen, V. M.; Andreassen, O. A. A possible association between schizophrenia and GRIK3 polymorphisms in a multicenter sample of Scandinavian origin (SCOPE). Schizophr Res 2009, 107, 242-8.
(143) Shibata, H.; Shibata, A.; Ninomiya, H.; Tashiro, N.; Fukumaki, Y. Association study of polymorphisms in the GIuR6 kainate receptor gene (GRIK2) with schizophrenia. Psychiatry Res 2002, 113, 59-67.
(144) Schiffer, H. H.; Heinemann, S. F. Association of the human kainate receptor GluR7 gene (GRIK3) with recurrent major depressive disorder. American Journal of Medical Genetics Part B-Neuropsychiatric Genetics 2007, 144B, 20-26.
(145) Peng, L.; Li, B.; Du, T.; Wang, F.; Hertz, L. Does conventional anti-bipolar and antidepressant drug therapy reduce NMDA-mediated neuronal excitation by downregulating astrocytic GluK2 function? Pharmacol Biochem Behav 2012, 100, 712-25.
(146) Samengo, I.; Curro, D.; Navarra, P.; Barrese, V.; Taglialatela, M.; Martire, M. Molecular and pharmacological evidence for a facilitatory functional role of pre-synaptic GluK2/3 kainate receptors on GABA release in rat trigeminal caudal nucleus. Eur J Pain 2012, 16, 1148-57.
(147) Larsen, A. M.; Bunch, L. Medicinal Chemistry of Competitive Kainate Receptor Antagonists. ACS Chemical Neuroscience 2010, 2, 60-74.

1. A compound of Formula (I) 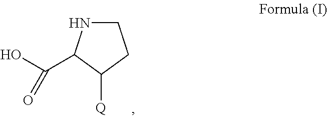
and pharmaceutically acceptable derivatives, as well as all tautomers and stereoisomers of compound of Formula (I), wherein 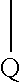
represents compounds of Formula (Ia) or (Ib); 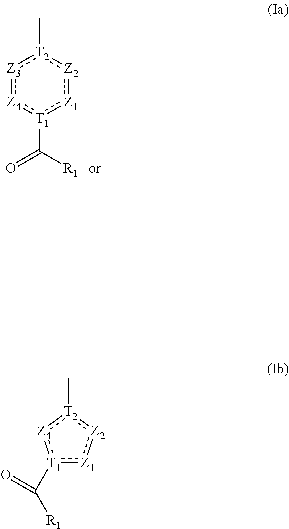
---- in each case may represent if appropriate the presence of at least one double bond between T₂ and Z₂, or between Z₂ and Z₁, or between Z₁ and T₁, or between T₁ and Z₄, or between Z₄ and (Z₃ or T₂), or between Z₃ and T₂; or
T₁ is C, CH,
T₂ is C, CH,
Z₁ is CR₂, C(R₂)₂, N, S, O, or NR₃,
Z₂ is CR₂, C(R₂)₂, N, S, O, or NR₃,
Z₃ is CR₂, C(R₂)₂, N, S, O, or NR₃,
Z₄ is CR₂, C(R₂)₂, N, S, O, or NR₃,
wherein the residues Z₁, Z₂, Z₃ and Z₄ can not represent adjacent O or S;
R₁ may together with Z₁ or Z₄, or
Z₂ may together with Z₁, or
Z₃ may together with Z₄,
form a saturated or unsaturated C₅- or C₆-cycloalkyl, or a saturated or unsaturated heterocyclyl containing 5 or 6 ring atoms, wherein the saturated or unsaturated C₅- or C₆-cycloalkyl, or the saturated or unsaturated heterocyclyl containing 5 or 6 ring atoms, may be substituted with one or more substituents selected from the group comprising OR₄ or R₄,
R₁ is H, OR₄, C₁-C₆-alkyl, C₂-C₆-alkenyl, C₂-C₆-alkynyl, COOR₄, N(OH)H, or NHR₄,
R₂ is independently selected among R₄, O, OR₄, halogen, N(OH)H, N(OH)R₄, NHR₄, COR₄, CONHR₄, or SO₂NHR₄,
R₃ is independently selected among R₄, O, OR₄, or halogen,
R₄ is independently selected among H, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine,
halogen represents Cl, Br, or I, and
with the proviso that Z₁, Z₂, Z₃ and Z₄ are not all CH, and T₁ and T₂ are not both C, when R₁ is OH.

2. A compound according to claim 1, comprising Formula (II) 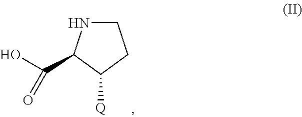
wherein 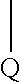
represents compounds of Formula (Ia1) or (Ib2); 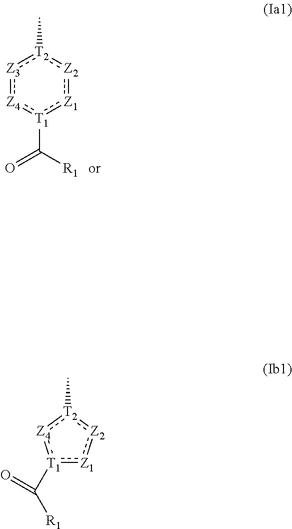
---- in each case may represent if appropriate the presence of at least one double bond between T₂ and Z₂, or between Z₂ and Z₁, or between Z₁ and T₁, or between T₁ and Z₄, or between Z₄ and (Z₃ or T₂), or between Z₃ and T₂; or
T₁ is C, CH,
T₂ is C, CH,
Z₁ is CR₂, C(R₂)₂, N, S, O, or NR₃,
Z₂ is CR₂, C(R₂)₂, N, S, O, or NR₃,
Z₃ is CR₂, C(R₂)₂, N, S, O, or NR₃,
Z₄ is CR₂, C(R₂)₂, N, S, O, or NR₃,
wherein the residues Z₁, Z₂, Z₃ and Z₄ can not represent adjacent O or S;
R₁ may together with Z₁ or Z₄, or
Z₂ may together with Z₁, or
Z₃ may together with Z₄,
form a saturated or unsaturated C₅- or C₆-cycloalkyl, or a saturated or unsaturated heterocyclyl containing 5 or 6 ring atoms, wherein the saturated or unsaturated C₅- or C₆-cycloalkyl, or the saturated or unsaturated heterocyclyl containing 5 or 6 ring atoms, may be substituted with one or more substituents selected from the group comprising OR₄ or R₄
R₁ may together with Z₁ or Z₄, or
Z₂ may together with Z₁, or
Z₃ may together with Z₄,
form a saturated or unsaturated C₅- or C₆-cycloalkyl, or a saturated or unsaturated heterocyclyl containing 5 or 6 ring atoms, wherein the saturated or unsaturated C₅- or C₆-cycloalkyl, or the saturated or unsaturated heterocyclyl containing 5 or 6 ring atoms, may be substituted with one or more substituents selected from the group comprising OR₄ or R₄.
R₁ is H, OR₄, C₁-C₆-alkyl, C₂-C₆-alkenyl, C₂-C₆-alkynyl, COOR₄, N(OH)H, or NHR₄,
R₂ is R₄, O, OR₄, halogen, N(OH)H, N(OH)R₄, NHR₄, COR₄, CONHR₄, or SO₂NHR₄,
R₃ is R₄, O, OR₄, or halogen,
R₄ is H, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine,
halogen represents Cl, Br, or I.

3. A compound according to claim 1, wherein 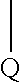
represents 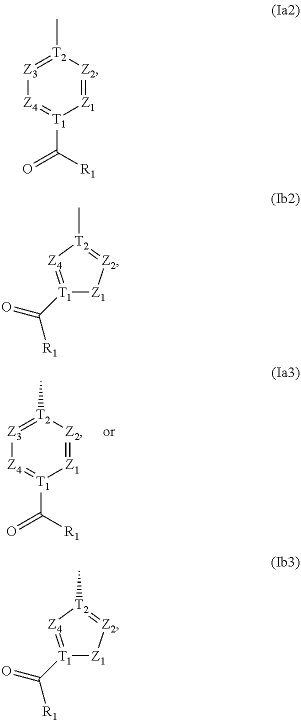
wherein
T₁ is C,
T₂ is C,
Z₁ is CR₂, N, S, O, or NR₃,
Z₂ is CR₂, or N,
Z₃ is CR₂, or N,
Z₄ is CR₂, or N,
wherein the residues Z₁, Z₂, and Z₃ cannot represent adjacent O or S;
R₁ may together with Z₁ or Z₄, or
Z₂ may together with Z₁ or Z₃,
form a saturated or unsaturated C₅- or C₆-cycloalkyl, or a saturated or unsaturated heterocyclyl containing 5 or 6 ring atoms, wherein the saturated or unsaturated C₅- or C₆-cycloalkyl, or the saturated or unsaturated heterocyclyl containing 5 or 6 ring atoms, may be substituted with one or more substituents selected from the group comprising OR₄ or R₄,
R₁ is H, OR₄, C₁-C₆-alkyl, C₂-C₆-alkenyl, C₂-C₆-alkynyl, COOR₄, N(OH)H, or NHR₄,
R₂ is R₄, O, OR₄, halogen, N(OH)H, N(OH)R₄, NHR₄, COR₄, CONHR₄, or SO₂NHR₄,
R₃ is R₄, O, OR₄, or halogen,
R₄ is H, C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl, wherein C₁-C₆-alkyl, C₂-C₆-alkenyl, C₂-C₆-alkynyl, phenyl, C₁-C₆-alkylphenyl, or saturated or unsaturated C₅- or C₆-cycloalkyl, or saturated or unsaturated C₁-C₆-alkyl C₅- or C₆-heterocyclyl may be substituted with one or more substituents selected from the group comprising C₁-C₆-alkyl, C₁-C₆-alkoxy, aryl, halogen, and amine,
halogen represents Cl, Br, or I.

4. A compound according to claim 1, wherein R₁ may together with Z₁ or Z₄, or if appropriate Z₂ may together with Z₁ or Z₃, form a saturated or unsaturated C₅- or C₆-cycloalkyl or a 5- or 6-membered heterocyclyl ring, the heterocyclyl ring containing one, two, three or four heteroatoms selected from the group comprising sulfur, oxygen and nitrogen.

5. A compound according to claim 4 wherein the 5- or 6-membered ring is selected from the group consisting of pyrrolidine, pyrrole, tetrahydrofuran, furan, thiolane, thiophene, imidazolidine, pyrazolidine, imidazole, pyrazole, oxazolidine, isoxazolidine, oxazole, isoxazole, thiazolidine, isothiazolidine, thiazole, isothiazole, dioxolane, dithiolane, triazoles, furazan, oxadiazole, thiadiazole, dithiazole, tetrazole, piperidine, pyridine, oxane, pyran, thiane thiopyran, piperazine, diazines, morpholine, oxazine, thiomorpholine, thiazine, dioxane, dioxine, dithiane, dithiine, triazine, trioxane, or tetrazine, and wherein the 5- or 6-membered heterocyclic ring may be substituted with 1 to 3 substituents.

6. A compound according to claim 4 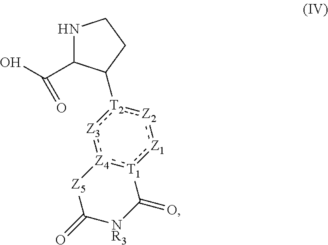
wherein
Z₅ is CR₂, C(R₂)₂, N, S, O, or NR₃,
wherein the residues Z₁, Z₂, Z₃, Z₄ and Z₅ cannot represent adjacent O or S.

7. A compound according to claim 4 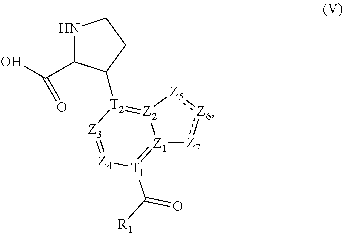
wherein
Z₅ is CR₂, C(R₂)₂, N, S, O, or NR₃,
Z₆ is CR₂, C(R₂)₂, N, S, O, or NR₃,
Z₇ is CR₂, C(R₂)₂, N, S, O, or NR₃,
wherein the residues Z₁, Z₂, Z₃, Z₄, Z₅, Z₆, Z₇ cannot represent adjacent O or S.

8. A compound according to claim 1, wherein R₂ is OR₄, halogen, N(OH)H, N(OH)R₄, NHR₄, COR₄, CONHR₄, or SO2NHR₄.

9. A compound according to claim 1, wherein R₄ is H, C₁-C₆-alkyl, C₂-C₆-alkenyl, C₂-C₆-alkynyl, C₁-C₆-alkylphenyl, wherein C₁-C₆-alkyl, C₂-C₆-alkenyl, C₂-C₆-alkynyl, C₁-C₆-alkylphenyl may be substituted with one or more substituents selected from the group comprising C₁-C₆-alkyl, C₁-C₆-alkoxy, aryl, halogen, and amine.

10. A compound according to claim 1, 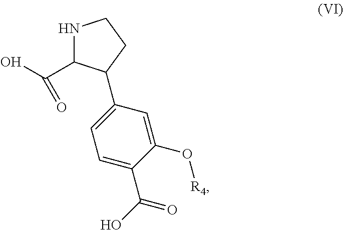
wherein
R₄ has the same meaning as given under claim 1.

11. A compound according to claim 10, wherein
R₄ is alkyl, benzyl, alkylbiphenyl, preferably propyl, benzyl, or 3-methyl[1,1′-biphenyl].

12. A compound according to claim 1 selected from the group consisting of
(1) (2S,3R)-3-(4-Carboxyphenyl)pyrrolidine-2-carboxylic Acid
(2) (2S,3R)-tert-Butyl 2-(((tert-butyldimethylsilyl)oxy)methyl)-3-(4-(((tert-butyldimethylsilyl)oxy)-methyl)phenyl)-5-oxopyrrolidine-1-carboxylate
(3) (2S,3R)-tert-Butyl 2-(((tert-butyldimethylsilyl)oxy)methyl)-3-(4-(((tert-butyldimethylsilyl)oxy)-methyl)phenyl)pyrrolidine-1-carboxylate
(4) (2S,3R)-tert-Butyl 2-(hydroxymethyl)-3-(4-(hydroxymethyl)phenyl)pyrrolidine-1-carboxylate
(5) (2S,3R)-3-(3-(Benzyloxy)-5-carboxyphenyl)-1-(tert-butoxycarbonyl)pyrrolidine-2-carboxylic acid.

13. A compound according to claim 1 for use as a medicament.

14. A pharmaceutical composition comprising a compound according to claim 1 in combination with one or more therapeutically acceptable diluents or carriers.

15. A compound according to claim 1 or a method for treatment of diseases or conditions binding one or more of the GluA1, GluA2, GluA3, GluA4, GluK1, GluK2, GluK3, GluK4, GluK5, GluN2A, GluN2B, GluN2C or GluN2D receptors subunits to obtain a beneficial therapeutic effect.

16. A method for treating a disease or disorder mediated by one or more of the GluA1, GluA2, GluA3, GluA4, GluK1, GluK2, GluK3, GluK4, GluK5, GluN2A, GluN2B, GluN2C or GluN2D receptor subunits, wherein the disease or disorder is selected from the group consisting of psychiatric diseases or neurological disorders or a disease or disorder associated with abnormal activities of one or more of the GluA1, GluA2, GluA3, GluA4, GluK1, GluK2, GluK3, GluK4, GluK5, GluN2A, GluN2B, GluN2C or GluN2D receptor subunits in a patient in need thereof, comprising administering to the patient a therapeutically effective amount of a compound according to claim 1, or a pharmaceutically acceptable salt or solvate thereof, optionally together with a pharmaceutically acceptable carrier.

17. A compound according to claim 1 or a method for treatment of disorders of the central nervous system, neuro-physiological processes such as memory, cognition; as well as neuronal plasticity and development, psychiatric diseases or neurological disorders such as depression, anxiety, addiction, pain, migraine, and schizophrenia, and neurodegenerative diseases; such as Alzheimer, Huntington disease, amyotrophic lateral sclerosis (ALS), cerebral stroke, and epilepsy; and diseases including aching, ADHD, Autism, Diabetes, Huntington's disease, ischemia, multiple sclerosis, Parkinson's disease (Parkinsonism), Rasmussen's encephalitis, seizures, AIDS dementia complex, amyotrophic lateral sclerosis, combined systems disease (vitamin B12 deficiency), drug addiction, drug tolerance, drug dependency, glaucoma, hepatic encephalopathy, hydroxybutyric aminoaciduria, hyperhomocysteinemia and homocysteinuria, hyperprolinemia, lead encephalopathy, leber's disease, MELAS syndrome, MERRF, mitochondrial abnormalities (and other inherited or acquired biochemical disorders), neuropathic pain syndromes (e.g. causalgia or painful peripheral neuropathies), nonketotic hyperglycinemia, olivopontocerebellar atrophy, essential tremor, Rett syndrome, sulfite oxidase deficiency, Wernicke's encephalopathy or cancer.

18. A pharmaceutical composition according to claim 14 wherein the pharmaceutical composition is administered in doses of 0.5-1500 mg/day, preferably 0.5-200 mg/day, more preferably 0.5-60 mg/day, even more preferably 0.5-30 mg/day.

19. A pharmaceutical composition according to claim 14 for oral, rectal, nasal, pulmonary, buccal, sublingual, transdermal or parenteral administration.
